(19) United States
(12) Patent Application Publication
(10) Pub. No.: US 2021/0161463 A1
(43) Pub. Date: Jun. 3, 2021

(54) SYSTEM FOR THE TELEMETRY MONITORING OF A PATIENT'S VITAL SIGNS AND TELEMETRY MONITORING METHOD

(71) Applicant: Facecontrol, Inc., Miami, FL (US)

(72) Inventors: Konstantin Nikolaevich Pronko, Moscow (RU); Andrei Borisovich Borsov, Moscow (RU); Alexandr Kusmich Volkov, Moscow (RU); Aleksei Nikolaevich Diashev, Moscow (RU); Vladimir Igorevich Sinopalnikov, Moscow (RU); Alexandr Alexandrovich Taranov, Moscow (RU)

(73) Assignee: Facecontrol, Inc., Miami, FL (US)

(21) Appl. No.: 16/632,504

(22) PCT Filed: Dec. 21, 2017
(86) PCT No.: PCT/EA2017/000004

§ 371 Date: Jan. 20, 2020

(30) Foreign Application Priority Data
Jul. 18, 2017 (EA) 201700326

(51) Int. Cl. A61B 5/00; A61B 5/16; A61B 5/103; A61B 5/11; A61B 5/0205

(52) U.S. Cl. CPC A61B 5/486; A61B 5/441; A61B 5/165; A61B 5/743; A61B 5/1107; A61B 5/0205; A61B 5/7264; A61B 5/1032

(57) ABSTRACT

The invention is directed toward allowing early detection of the onset of functional disorders, correcting same and monitoring the effectiveness of prophylactic measures on the basis of the asymmetry of microcirculation in the blood vessels of the face and the contraction of the muscles of facial expression. This technical result is achieved in that a system for the telemetry monitoring of a patient's vital signs comprises devices for the measurement, input and reading of signals, parameters and graphical parameter data, which make it possible to monitor the parameters of an asymmetric change in skin colour in areas of the patient's face. A method for using this system includes determining primary physiological parameters, evaluating data obtained and analyzing and correcting same, said parameters being determined by recording, on projections of the skin of a patient's face, a change in the colour gradient of the skin and a vector change in monitoring points of the muscles of facial expression, wherein the patient looks at an image of themselves on a graphical data display means for a set period of time, data obtained about the colour of elements of the image and also a colour synchronizing signal are received by a signal processing system, and the signals are evaluated taking into account set/known coefficients of change in colour and coordinates for determining the required parameters.

### FIELD OF THE INVENTION

The proposed invention pertains to medicine, more specifically, the invention relates to non-contact telemetric personified predicative preventive diagnostic apparatus and is intended for early detection of risks of functional disorders, correction of psychosomatic disorders, and morphological changes in the human body.

The capabilities of current microcirculation test methods allow non-invasive assessment of fundamental physiological processes in the body and obtaining data that were previously beyond reach.

The development of the biomedical telemetry market is aimed at mastering methods of intellectual data analysis, including predictive variable models, simulators, and decision-making support systems.

The proposed invention enables to assess the vasomotor activity of resistive precapillary arterioles and precapillary sphincters, the exchange surface area, and the efficiency of the filtration-reabsorption metabolic mechanism directly related to the microcirculation parameters and enables the exchange of water-soluble and low-molecular weight substances.

The known method of applying predictive analytics includes the use of screening models for assessing the functional state. The current technological development in neurophysiology and bioelectronic medicine enables a new look at the capabilities of test systems for assessing human adaptive and psychophysiological abilities.

The screening test to diagnose obstructive sleep apnea is a case in point. The Republic of Kazakhstan, innovation patent number: 30738 dated Dec. 25, 2015.

Method of rapid assessment of arterial vasculature functional state, patent of the Russian Federation 2523680

The applicant has not identified any close equivalents of the proposed invention in the stated technical field.

The technical effect achieved through the use of the proposed invention provides early identification of functional disorder risks, their correction and monitoring (control) of the efficiency of preventive measures based on facial microcirculation asymmetry and facial muscle contractions.

The aforesaid technical effect is achieved by the fact that the telemetry control system for patient's vital functions contains apparatus for measuring, input and reading of signals, parameters, and graphic data of parameters, including coordinate apparatus based on computer vision technologies that allow monitoring of asymmetric skin color change parameters on areas of the patient's face, a processor which is connected to the apparatus for processing the recorded parameters, and an apparatus for displaying the graphic data of the patient's image. The processor is designed so as to process the input signals based on the preset color change coefficients to determine the optical density of tissues, plethysmographic data, and oxygenation parameters.

The apparatus for input and reading of signals, parameters, and graphic data of parameters is made with the possibility to monitor the skin color gradient with a frequency of at least 30 frames per second.

The apparatus for input of graphic data with the processor connected to it is made with the possibility to use computer vision to ensure coordinate control of facial areas and facial contractions.

The processor allows you to determine the main waves to control the patient's vital functions after processing a signal and create a control program based on the principles of biofeedback. The system of the processor provides the ability to output graphic and acoustic signals to influence the patient in order to correct his/her functional state.

The claimed method for telemetric monitoring of patient's vital functions using the system, including determining the basic physiological parameters, assessing and analyzing the obtained data, as well as correcting the patient's functional state; these parameters are determined using the graphic information input apparatus and the processor by tracking and recording changes in the color chromaticity gradient and vector changes at the control points of the facial muscles using projections of the patient's face; at the same time, the patient looks at his/her own image on the display/screen of the computing apparatus with the means for displaying graphical information for a predetermined time, after which the obtained data on the color of the image elements, as well as the color synchronization signal, enter the signal processing system, and the system evaluates the signals taking into account the specified/known coefficients of changes in the chromaticity and coordinates to determine the required parameters. The system records the signals of changes in the color of the image elements and vector changes in the coordinates of the control points in at least 2 areas of the patient's face skin at 12 control points; at the same time, the patient looks at his/her own image on the display/screen of the computing apparatus designed to display graphic information for at least 300 seconds, which corresponds to the minimum time interval required for assessing human heart rate variability.

The data received from the input apparatus and processed by the processor's program allow to determine the amplitude parameters of the oscillations of slow physiological waves of a human, with high accuracy: pulse waves, respiratory waves, as well as waves that are caused by parasympathetic or sympathetic cholinergic influences, the myogenic activity of microvascular myocytes, the influence of sensory peptidergic nerve fibers on neuropeptide myocytes, the low-frequency rhythm of impulses of sympathetic adrenergic vasomotor fibers, and the effects of endothelial nitric oxide. Recording of signals related to changes in the color of the elements of the image of the patient's face skin and vector changes in the coordinates at control points from the projection of the patient's face makes it possible to determine blood perfusion in areas of no more than 2 square centimeters. In addition, recording of parameters related to changes in the color of image elements and vector changes in the coordinates at control points from the projection of the patient's face allows to identify the asymmetry of parameters: amplitudes of oscillations of slow physiological waves and manifestations of emotionally conscious and unconscious micromimic facial expressions that may be used by the system of telemetric detection to assess the reliability of transmitted information.

The claimed invention is a system and a method for early detection of risks of functional disorders, correction of psychosomatic disorders, and morphological changes in the human body, their correction and monitoring (control) of the efficiency of preventive measures taking into account the parameters of the amplitude of oscillations of physiological slow waves, the asymmetry of microcirculation of blood through the vessels, as well as conscious and unconscious facial muscle contractions.

Taking into account the above and other considerations and in accordance with the claimed invention, we have provided a graphic information input apparatus and a processor for implementing a method for early detection of the risks of functional disorders, their correction and monitoring (control) of the effectiveness of preventive measures. The method involves using a computer program for executing commands stored in memory using at least one processor of an electronic apparatus to receive data describing human heart rate variability while simultaneously recording and processing the elements of the resulting image and tracking changes in the color of the face skin, taking into account the specified algorithms.

The method further provides the possibility of filming a person's face for a predetermined period of time using the digital camera of the electronic apparatus. Images and videos are processed to track changes in the skin color gradient and to monitor the patient's facial muscle contractions.

Furthermore, the method includes recording a video of a person using a digital camera with a resolution of at least 640×480 pixels, dividing the image elements into individual frames, determining the elements of the face, filtering the skin color according to the specified conditions, coordinating distribution of the boundaries of areas that are subject to investigation, calculating averages for the channels of the additive RGB color model, noise filtering, and drawing a cardiointervalogram.

The claimed system includes a monitoring module for conscious and unconscious facial muscle contractions, thus allowing to evaluate the physiological mechanisms of the individual stages of the information processing process: sensory analysis, activation of attention, formation of images, retrieving of memory standards, decision making, etc.

The set of computer commands includes commands to turn on the graphic information input apparatus and record image parameters, including coordinate apparatus that use computer vision to record a video of an image of a person's face for a specified period of time. Then, the recorded video is processed to assess the parameters of vital functions and create programs for correcting the person's functional state.

The system uses a processor with a software-mathematical algorithm and allows to determine the main waves in the processed signals to control the patient's vital activity and create programs for correcting the functional state of the body based on biological feedback, taking into account the radiation characteristics of the display (monitor); an acoustic system and other peripheral apparatus are used to influence the patient in order to correct his/her functional state.

The method for telemetric monitoring of patient's vital functions using the claimed system includes determining the basic physiological parameters, assessing and analyzing the data, as well as correcting the patient's functional state. In this case, the parameters are determined using the image input apparatus and the processor by recording changes in the skin color of the patient's face and facial muscle contractions, while the patient is looking at his/her own image on the monitor for a specified time. The obtained data can then be processed directly by the processor or transmitted via the Internet to the server for signal processing, and the signals are evaluated according to the requirements set by the operator (doctor), otherwise the predetermined algorithm for a particular patient or standard methods are used.

The claimed method can also be implemented as a mobile application for the telemetric monitoring system that allows you to evaluate the characteristics of the measurements obtained in the remote access system, compare it with the previously obtained data, and provide the decision support model to the doctor when assessing the patient's health status with the aim of prescribing a therapeutic treatment.

If necessary, the program allows you to generate external stimuli and evaluate the human (patient's) reactions to them in order to assess his/her psycho-emotional state. For example, psychological tests, game tasks to evaluate the sensory-motor response, physical activity, etc., can be used; all actions and reactions of a person are automatically controlled; the system tracks physiological parameters such as pulse, respiration, second-order waves from the description above.

Generating external stimuli and tracking of the facial microexpressions, including asymmetry of microexpressions of a patient in the process of recording parameters using the graphic information input apparatus and analyzing them using the program, taking into account at least nine control points, allow you to evaluate the physiological mechanisms at the individual stages of the information processing: sensory analysis, activation of attention, formation of images, retrieving of memory standards, decision making, etc.

The claimed method allows you to monitor the micromimic contractions of facial muscles controlled by the somatic nervous system (cranial nerves); these processes involve the sensory and motor nerves of the muscles and skin that belong to the parasympathetic nervous system. The parasympathetic nervous system also includes the facial nerve (facial muscles) and the ocular motor nerve (eyeball and eyelids).

In order to reliably estimate facial muscle contractions by vector analysis, the coordinates of the control points of the face image must be recorded at intervals of less than 0.05 seconds.

The graphic information input apparatus records color gradient changes, i.e., each pixel of the frame with the face skin changes color when the pulse wave propagates.

The graphic information display apparatus can be used to determine the mobility (lability) of muscular and nervous tissues in the presence of a source of liminal irritation in the form of light (color) stimulation. For example, to assess the lability of vegetative fibers, the program uses the graphic information display apparatus to generate a red rhythmic stimulus of maximum brightness with a frequency of 200 pulses per second.

Synchronized assessments of the microcirculation in blood vessels, the micromimic processes of pain sensitivity control, and the effects of cold allow to determine the proportion of participation of each action and calculate the correlation between emotional and psycho-physiological reactions. The program also includes a method for assessing the correlation between data from various actions which allows you to distinguish between individual spontaneous unconscious emotionally significant microexpressions and feelings that are expressed by facial muscles.

The need to evaluate at least two areas of the facial skin is determined by the microcirculation system. The microcircular vasculature that comprises repetitive functional units, modules, each of which is a specific multicomponent complex consisting of nerve conductors, organ cells, and microvessels (arterioles, precapillary arterioles, capillaries, postcapillary venules, collecting and other venules, arteriolo-venular anastomoses, and lymphatic vessels). This complex of functional structures ensures the maintenance of homeostasis and blood-lymphatic balance. Each microcirculatory module is separated from the neighboring ones both structurally and functionally, since it has isolated pathways of inflow and outflow of blood and tissue metabolism products. Algorithms of mathematical analysis implemented in this complex are used to compare the microcircular modules of facial projections and provide a significant diagnostic feature.

The graphic information input apparatus and the processor allow to evaluate changes in muscle activity, neural signals, and rheographic parameters of blood when recording afferent (sensory) and efferent (motor) reactions in the range of physiological stimuli of different directions that are generated using the graphic information display apparatus. Evaluation of the recorded indicators allows to prepare individual recommendations for the user in his/her daily activities, eliminating the imposed socio-cultural behavior related to preferences in the field of nutrition, sports, sex, music, recreation, entertainment, clothing, shopping, marketing, and other areas of human life.

Thus, the method of assessing the state of the mechanisms of regulation of physiological functions in the human body, in particular, the general activity of regulatory mechanisms, neuro-humoral regulation of the heart, relationships between the sympathetic and parasympathetic parts of the autonomic nervous system, involved the use of standard statistical methods and algorithms for mathematical analysis. To assess the reliability, the effects obtained were compared with the effects obtained by standard exposure diagnostic methods; see example in the next section.

The claimed invention is confirmed by the following examples.

### EXAMPLE 1

1. Verification of the reliability of the effects obtained using the telemetric monitoring system and the method of obtaining parameters of vital human functions, as well as the correlation between the physiological parameters of trial subjects recorded using the telemetric monitoring system and standard diagnostic methods.
2. Sixty subjects took part in the trials: 30 trial subjects were considered conditionally healthy, and 30 trial subjects received a confirmed diagnosis of ICD-10 (F45.3) somatoform autonomic dysfunction.
3. All the trial subjects were given Korean-made tablets, equipped with a webcam, microphone, and Internet access. The tablets also had a pre-installed program of the telemetric monitoring system of parameters of vital human functions with Internet access and access to the server of the telemetric monitoring system.
4. In order to confirm the reliability of data obtained using the telemetry system, the following standard diagnostic apparatus and methods were used in the trials:

Electrocardiograph (ECG)

Doppler ultrasound (USDG)

Tissue oximeter: OxiplexTS, ISS Inc., USA

Magnetic cardio-encephalograph (MCEG)

Photoelectric pulse oximeter
5. Parameters of chronobiological oscillatory components and constants of vital functional parameters of self-regulation of organs and body systems were subject to assessment:

Heart rate, variational pulsometry and oximetry

Slow heart rate waves: ULF, VLF, LF

Respiratory waves
6. The data obtained as an effect of the research confirmed the high informativity of the created system and the developed methods for diagnosing the functional state and allowed to create algorithms for correlation with standard clinical methods.
7. The goal of the second stage of the research was to assess the possibility of using biofeedback methods to correct the functional state of 30 subjects who were diagnosed with somatoform autonomic dysfunction.
8. Each trial subject had their heart rate variability parameters recorded for 600 seconds in a sitting position. The data obtained were evaluated by a cardiologist.
9. After that, the Stange's tests were carried out, and the average inspiration breath hold for all the trial subjects did not exceed 30 seconds.
10. In order to increase adaptive capacity, trial subjects were asked to conduct autogenic respiratory training for 9 minutes with a motivated control of their own pulse and respiratory waves on the graphical display of the telemetric control system.

11. At 60 minutes after the training, Stange's tests with the use of biofeedback demonstrated an increase in average breath hold of trial subjects to 40 seconds.

### EXAMPLE 2

1. Online telemedicine consultation using the telemetric control system for parameters of vital functions with the patient V. F., born in 1931, diagnosed with hypertension and group 2 dissability, using remote access to the Internet.
2. The patient's call via the Internet for consultation with the attending physician about a hypertensive crisis that occurred one hour before the call. Standard recommended drugs did not stop the crisis.
3. During the consultation, the parameters of heart rate variability, pulse, and respiration were recorded using 5 projections of the face, and micromimic expressions were recorded using projections of points 1-12. Please see FIGS. 1 and 2 respectively.
4. The telemetric control system allowed real-time evaluation of the actual parameters of the cardiovascular system at the time of the call and helped correct the pharmaceutical therapy. The recommended drug and psychotherapeutic conversation in the format of a 15-minute consultation allowed to stop the crisis, and the normalization of the heart rhythm and pressure parameters were confirmed during the second consultation that took place in 2 hours.

### EXAMPLE 3

1. Online telemedicine consultation using the telemetric control system for parameters of vital functions with the patient K., born in 1958, suffering from pain in the mediastinum, loss of strength, and nausea, using remote access to the Internet.
2. During the consultation, parameters of heart rate variability and respiration were recorded using 5 projections of the face. Please see FIG. 1.
3. Micromimic expressions were also evaluated using projections of points 1-12. Please see FIG. 2.
4. The system allowed real-time evaluation of the actual parameters of the cardiovascular system at the time of the call and the asymmetry of movement of the blood vessels of the face and facial muscles in the process of taking patient history. A decision support system, which is part of the web-based parameter analysis application, provided recommendations for urgent hospitalization to prevent transient cerebral ischemia. The consulting physician made a decision and recommended urgent hospitalization. The decision made by the system and the preliminary diagnosis of the transient-ischemic attack in the vertebrobasilar basin were confirmed in the hospital.

### BRIEF DESCRIPTION OF FIGURES

The accompanying figures/drawings with the same reference numbers refer to identical or functionally similar elements of all individual types and are included below with a detailed description as part of the specification; they serve as additional illustrations of the various embodiments and help explain the various principles and advantages in accordance with the claimed invention.

FIG. 1. A block diagram of an exemplary distributed data processing network using a personal mobile computing device, a personal computer, and a server/database in connection with the application of the claimed invention.

FIG. 2. A block diagram of an illustrative electronic apparatus, such as a personal mobile computing device, in accordance with the claimed invention.

FIG. 3. A block diagram of the process of telemetric monitoring of parameters of the vital functions of the patient in accordance with the aspect of the claimed invention.

FIG. 4. A schema of face image clustering and cardiointervalogram formation in accordance with the aspect of the claimed invention.

FIG. 5. A schema of the possible location of a user relative to a personal computing apparatus in accordance with the aspect of the claimed invention.

FIG. 6. A screenshot showing a set of the main waves when controlling the patient's vital functions obtained in accordance with the aspect of the claimed invention.

FIG. 7. A block diagram of a set of logical structures that implement various stages of the process, corresponding to the application of the claimed invention.

FIG. 8. A diagram of the coordinate distribution of control points, the vector displacement of which is one of the diagnostic features in accordance with the aspect of the claimed invention.

FIG. 9. A screenshot illustrating the possibility of estimating the parameters of the pulse wave by monitoring the nail phalanx of a finger in accordance with the aspect of the claimed invention.

The claimed invention is a new and effective computer-based system and method for objectively studying the state of the human body using telecommunication and information technology tools to provide clinical health care from a distance.

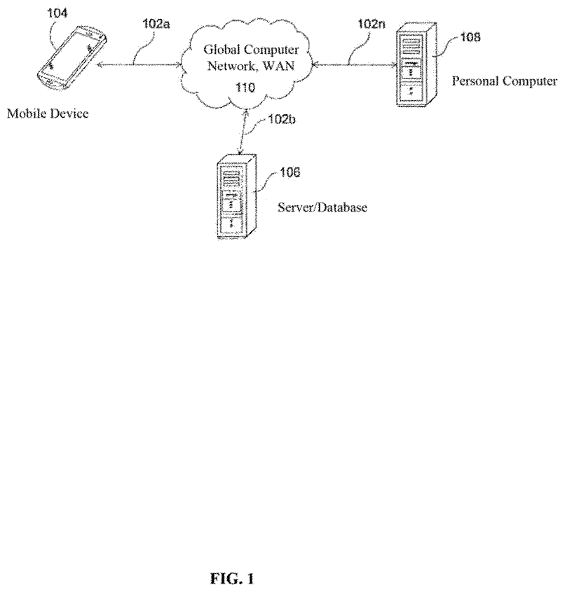

FIG. 1 shows one of the embodiments of the claimed invention in the form of a block diagram illustrating an exemplary network of a data processing system in which the claimed invention may be implemented. FIG. 1 illustrates some of the advantages of the claimed invention, however, as described below, the invention itself can be embodied in several shapes and sizes with different combinations of properties and elements and different numbers of components and their functions. The first example of network 100, as shown in FIG. 1, includes 102a-n connections, which are the medium for communication between various apparatus and computers interconnected in network 100. Connections 102a-n may be wire or wireless. Examples of wired connections are: cable, telephone line, and fiber optic cable. Examples of wireless connections are: radio frequency (RF) and infrared (IR). Many other wired and wireless connections are known in this technical field and can be used concurrently with the claimed invention.

In this example, the network 100 includes an electronic apparatus, such as a personal mobile computing device 104, a server 106, and a personal computer 108. The personal mobile computing device 104 may be used to execute programming commands contained in software that can be obtained from server 106 via WAN 110. In other embodiments of the invention, a personal computer 108 may execute programming commands received from the server 106 over the WAN 110. In other embodiments, the software is a web application, desktop software, or a mobile application. In one embodiment, the WAN is the Internet. The Internet is a worldwide collection of networks and gateways that use the TCP/IP protocol suite to communicate with each other. The Internet is based on a unifying backbone consisting of high-speed data lines between the main nodes or host computers represented by thousands of commercial, government, educational, and other computer systems that route data and messages. Of course, network 100 may also be implemented as many different types of networks, such as, for example, an intranet, a LAN, or a cellular network. FIG. 1 is considered as an example rather than a structural limitation for the claimed invention.

The server 106 can be considered as a computer that controls access to the centralized resource or database. In some embodiments of the invention, users of the personal mobile computing device 104 may request the software, which is an example of the use of the claimed invention. The server 106 can receive, process, and execute requests by transmitting the software to the personal mobile computing device 104 via WAN 110. In other embodiments, the personal computer 108 may request the software, and the server 106 may receive, process, and execute the request by transmitting the software to the personal computer 108 via WAN 110.

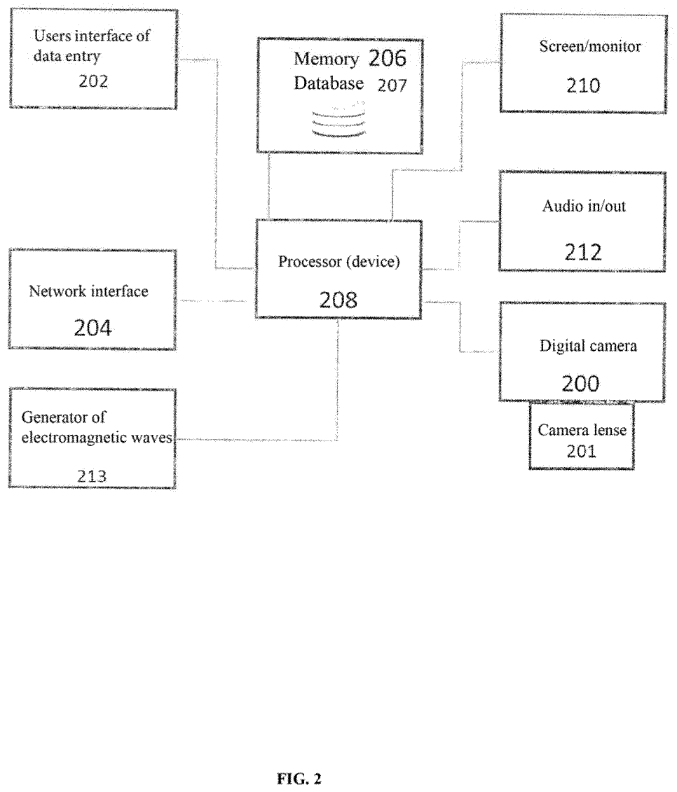

Now we will discuss FIG. 2. A Personal computer 104 is illustrated in the block diagram. The personal computer 104 has the following components: camera 200, user input interface 202, network interface 204, memory 206, processor 208, computer display 210, audio input/output 212, and generator 213.

The Camera 200 includes a camera lens 201, and it can be used to record still images and videos. The Camera 200 should preferably be digital, so that the images can be stored in memory 206 and processed by the processing apparatus 208. The Camera 200 is connected to a microphone for recording sound, including simultaneously with images. The Camera 200 is preferably used to record images with a resolution of at least 640×480 pixels to allow accurate interpretation and analysis of images in accordance with the methods described herein, as well as methods well known in this technical field. Lower quality cameras may not be able to shoot high-resolution images. The user input interface 202 provides user input to the personal computer 104. The user input interface 202 may also facilitate interaction between the user and the apparatus 104.

The user input interface 202 is a keyboard that supports a variety of user input operations. For example, the keyboard may include alphanumeric keys for entering (for example, phone numbers, contact information, text, etc.). The user input interface 202 may include special function keys (for example, to release the camera shutter and adjust the volume, the Back button, the Return button, etc.), as well as navigation and selection keys, the cursor, and so on. Keys, buttons, and/or keyboards may be implemented as a touch screen associated with the display of the computer 210 of a type known in this technical field. The touch screen can also provide data output or feedback to the user, for example, tactile feedback or adjusting the keyboard orientation in accordance with the signals of motion sensors, such as an accelerometer located inside the apparatus 104.

The network interfaces 204 may include one or more network interface cards or a network controller. In some embodiments, the network interface 204 may include PAN, a personal area network interface. The PAN interface may allow the personal computer 104 to connect to the network using a short-range data transfer protocol, such as Bluetooth. The PAN interface allows one personal computer 104 to establish a wireless connection with another personal computer 104 using a peer-to-peer connection.

Network interfaces 204 may also include LAN, a local area network interface. The LAN interface can be, for example, a wireless LAN interface, including Wi-Fi. The range of the LAN interface usually exceeds the range available for the PAN interface. In most cases, a connection between two electronic apparatus via a LAN interface may include a network router or other intermediate apparatus.

In addition, network interfaces 204 may include connections to a global WAN through a global WAN interface. The WAN interface can provide connectivity, for example, via cellular networks. A WAN interface can include elements such as an antenna connected to a radio circuit having a transceiver apparatus for transmitting and receiving radio signals using the antenna. The radio circuit can be configured to operate in a cellular network, including, but not limited to, GSM global mobile communications systems, CDMA code division multiple access, wideband CDMA, etc.

The personal computer 104 may also include an NFC short-range communication interface. The NFC interface can provide extremely close communication range at relatively low data transfer rates (for example, 424 kbps). NFC technology is based on the principle of magnetic field induction, which allows the NFC interface to interact with other NFC interfaces located on other mobile devices 104, or retrieving information from tags with embedded RFID identification schemes. The NFC interface can provide activation and/or acceleration of data transfer from one personal computer 104 to another personal computer 104 with an extremely close range (for example, 4 centimeters).

The memory 206 associated with apparatus 104 may be, for example, one or more buffers, flash memory, or non-volatile memory, including random access memory RAM. The personal computer 104 may also include non-volatile memory. The non-volatile memory may be any suitable storage medium, such as a hard disk or non-volatile memory, flash memory in particular. The memory 206 may include at least one database 207, which will be described in more detail below; this database is connected to the data apparatus 208 of the mobile device 104. In an embodiment of the invention, where the database 207 is considered to be at least a part of the memory 206 of the personal computer 104, such a communication link may be a hard wired connection. In an embodiment of the invention, where the remote database 106 is considered to be the database 207, accessible via, for example, long-distance networks, such as WAN 110, such a communication link can be established through the network interface 204 on the mobile device 104. The term “database” is broadly used for an ordered set of data that is stored in non-volatile memory and is available for a data processing apparatus that uses the set of data to solve tasks determined by a computer.

The data processing apparatus 208 may be, for example, a central processing unit, a microcontroller, or a microprocessor apparatus with a “general purpose” microprocessor or a “special purpose” microprocessor.

The processing unit 208 executes the code stored in the memory 206 to execute operations/commands from the personal mobile computing device 104. The data processing apparatus 208 may provide processing capability for operating system management, running various applications, and processing data for implementing one or more of the methods described herein.

The computer display 210 displays information for the user, including operating status, time, phone numbers, various menus, application icons, pop-up menus, and so on. The computer display 210 may be used to display various images, text, graphics or videos, in particular, photographs, mobile television content, web pages, and mobile application interfaces for the user. One example of the configuration of the display 210 is displaying a user cardiointervalogram as is described below. The computer display 210 may be any type of suitable display, including a liquid crystal display, a plasma display, a LED display, etc.

The personal computer 104 may include audio input and output components 212, such as a microphone for receiving audio signals from the user and/or a loudspeaker for playing audio signals, such as audio recordings associated with the user's speech and/or any sounds, movements, etc. The personal computer 104 may also include an audio port for peripheral audio input and output components, such as a headset, peripheral speakers, or microphones.

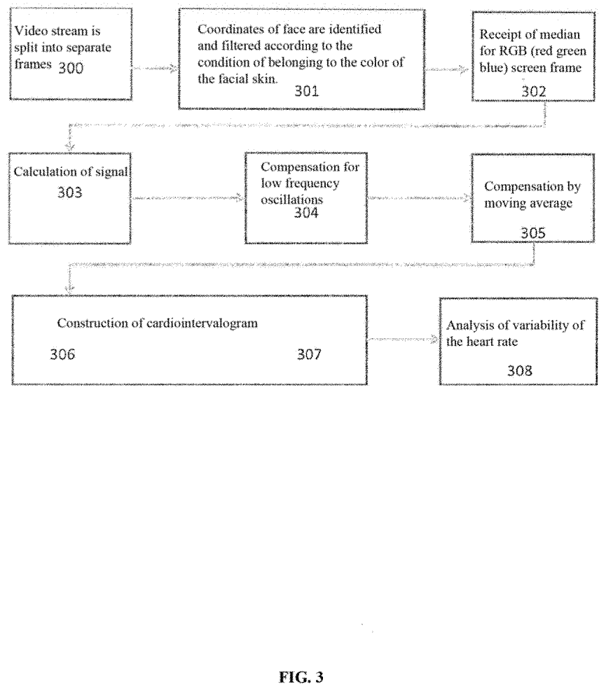

FIGS. 1-2 and 4-9 will be described in connection with the process flow diagram shown in FIG. 3. Although FIG. 3 shows the specific order of the steps in the process, the order may be changed and may not correspond to the order used in some embodiments of the invention. In addition, two or more blocks depicted sequentially can be executed simultaneously or with partial coincidence in some embodiments. To make the description shorter, some steps can also be omitted in FIG. 3. In certain embodiments of the invention, some or all of the process steps shown in FIG. 3 can be combined into one process.

The process example shown in FIG. 3 begins at step 300 and continues to step 308, where the user instructs the system to begin determining heart rate variability parameters. The user's command to start is received through the user interface 202 on the personal computing apparatus 104. A Start or Launch button can be made available to the user so that he/she can make a choice and confirm that he/she is ready to begin the process of determining the cardiointervalogram parameters. The user does not change position relative to the camera 200 for a certain period of time, as a rule, not less than 30 seconds, or for a sufficient time to determine the parameters of the heart rhythm, which is designated as the “determination period”. It should be clear to those skilled in the technical field that there are a number of other ways for the user to send a “start” command or another launch command, in particular, a voice signal recognition command, or other methods/structures for entering a user message into the personal computer 104. In accordance with an additional option, after the user sends a command to start the cardiointervalogram determination period, the display 210 and/or the audio output 212 may replay a countdown or otherwise prepare the user to take the correct position opposite the camera before the start of the determination period. As for the exemplary embodiment, the display 210 counts down from 10 to give the user 10 seconds to take the correct position. It is important that the user is properly positioned relative to the camera 200, and the camera is able to capture the user's image/video during the determination period. Tests have shown that serious interference that distorts the user's face image, such as thick glasses, will adversely affect the accuracy of the measurements. Therefore, it is preferable that the user removes such obstacles for facial analysis during the entire determination period.

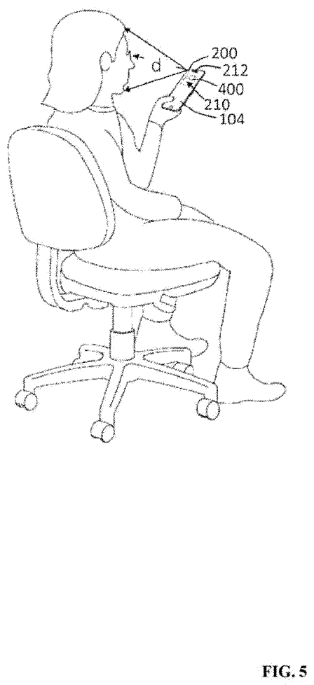

During the determination period, the user must be at exactly the same distance from the lens 201 of the camera 200. In one embodiment, the personal computer 104 may prompt the user to be located opposite the lens 201 of the camera 200 within one separation distance d, as shown in FIG. 5. The personal computer 104 may advise the user, for example, by means of a visual message 500, to “move closer,” “move further,” “stay in the same position,” or generate other similar messages that are displayed on the display 210 and/or reproduced as an audio signal using the audio output 212 of the personal computer 104 to ensure that the user remains at a predetermined separation distance optimal for the camera 200 to record images/videos for subsequent processing.

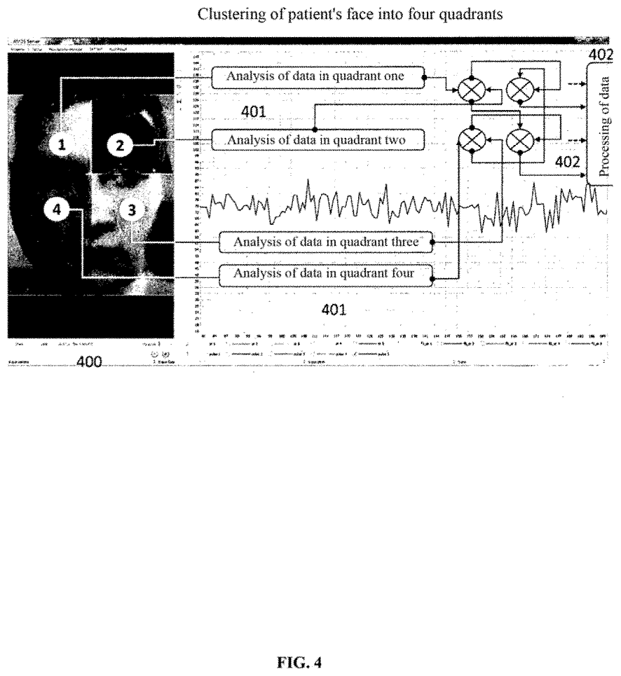

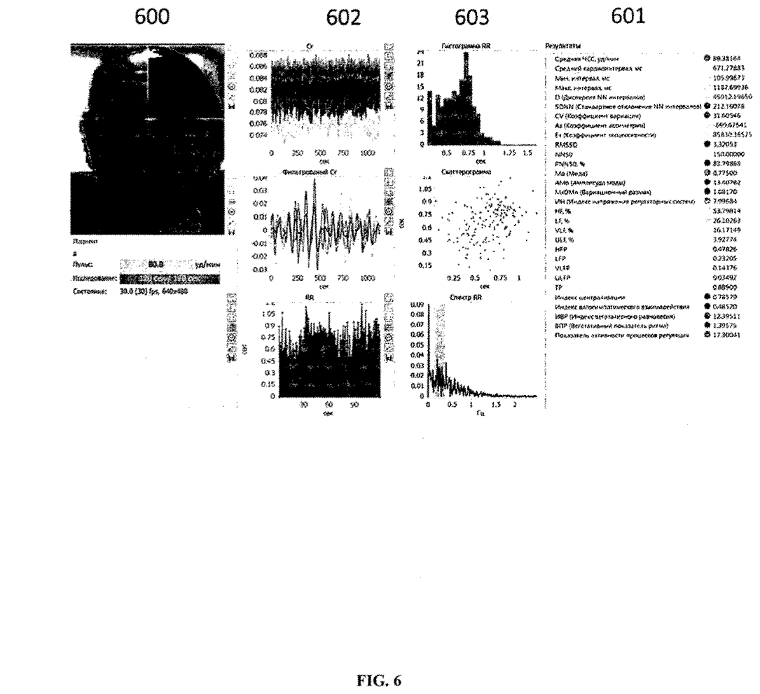

In order to visualize the stages of converting graphic information from the input apparatus 200 to the parameters of vital functions of the human body 308 used by the claimed invention, the stages of this process are presented in the block diagram. At step 300 (FIG. 3), the video stream is split into separate frames; at step 301 in the apparatus 208, each pixel of the obtained image is filtered according to the condition of belonging to the color of the facial skin, and the system determines coordinates of the boundaries of the areas to be studied; at step 302, the apparatus 208 calculates the average value for changes based on the additive RGB color model using clustering parameters (FIG. 4) for each frame; at step 303, the parameters of dynamic changes in the video stream are calculated based on the significance of the parameters of the additive color model for solving the diagnostic problem; FIG. 6 shows an example of the calculation for the Cr component video signal to assess the frequency of external respiration and the parameters of second-order waves associated with this function; at stages 304 and 305, the system filters noise and compensates for low-frequency oscillations associated with the movement of the head; at steps 306-308, the apparatus 208 calculates the basic parameters of heart rate variability and builds a cardiointervalogram easy to understand and evaluate by the user and the healthcare professional using the telemedicine system.

One of the important distinctive features of the claimed invention is the clustering of the parameters of the image of a face obtained from the camera 200 or apparatus 104 by the processor 208 using at least two projections; FIG. 4 shows the clustering of the patient's face into four quadrants 400, while the data processing is based on the mandatory assessment of the relationship between asymmetric changes in the color of the skin 402 and analysis of data on the optical density of tissues, plethysmography and oxygenation, as well as constructing the cardiointervalogram 401.

In order to record reliable graphic information that allows monitoring parameters of vital functions of the human body, let us again consider FIG. 5, where the user has a graphic information input apparatus 104, for example, with a mobile phone and a built-in digital camera 200, at a focal distance d for convenience while holding it in a bent arm, or a personal computer 108 with an integrated web camera 200 with mandatory coverage of the user's face, at least 70% of the visual display 210. The claimed invention provides for the possibility of recording data in any convenient (necessary) position of the patient, namely, sitting, lying, standing, or during physical activities.

In order to ensure the visibility of the heart rate variability data obtained after conversion, FIG. 6 shows patient data 600 including statistical 601, geometric 602, and spectral 603 assessment data; if the user agrees, these data can be transferred to a medical consultant for analysis and practical recommendations using the network resource 110. The claimed invention provides for teleconsultations by healthcare professionals and psychologists, while ensuring the safety of the use of databases 207 and the accumulation of a large amount of patients' personal data on the server of the developer 106; in addition, the presented data are compared with previous measurements.

In order to use diagnostic methods and practices of interviewing and collecting patient history data, the claimed invention provides for the possibility of synchronized input and output 212 of audio signals. The invention also provides the possibility of using the obtained data for the correction of the patient's psycho-physiological functions using biological feedback; to this end, the processor can be connected to any apparatus 214 that provides electromagnetic, bioelectronic, quantum, acoustic, or other synchronized (modulated) effects for the parameters of the patient's vital functions.

The algorithm for synchronized control of the asymmetry of oscillations of human physiological slow waves and emotional motor manifestations in the invention can be used by the user and/or consultant for psychological testing and assessment of psychosomatic conditions.

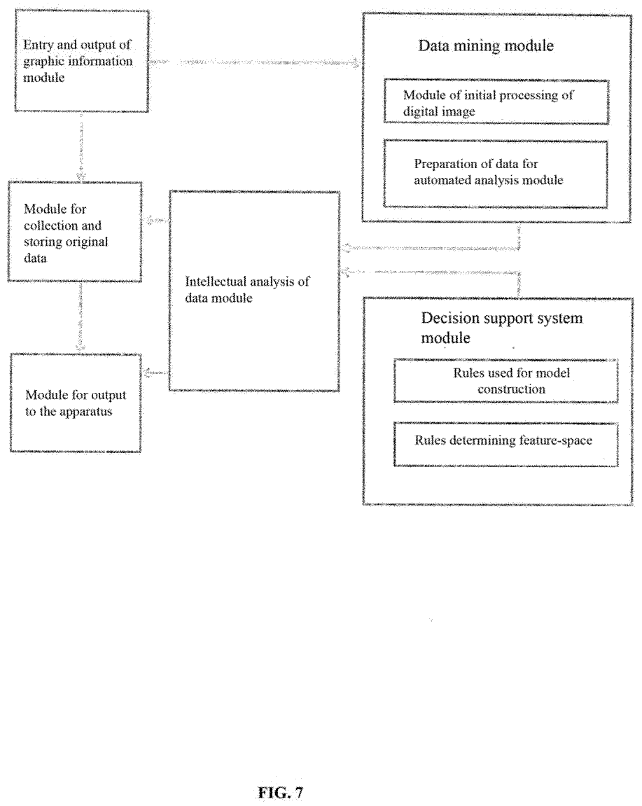

FIG. 7 shows a block diagram of a set of logical structures that implement various stages of the process, corresponding to the application of the claimed invention. The block diagram illustrates the possibilities for transmitting data from the graphic information input and output module 701 of the electronic apparatus 104, transmitting data to the module for collecting and storing the original data 702 included in the system memory 206 for long-term storage and the module for collecting and processing information 703 incoming to system 208 for the purpose of transmitting data for processing in the data mining module 704 and further comparing and analyzing data from the decision support system module 705. After comparing and analyzing the data, the effects can be transmitted using the output module 706 to the apparatus 106 or via the WAN 110 networks.

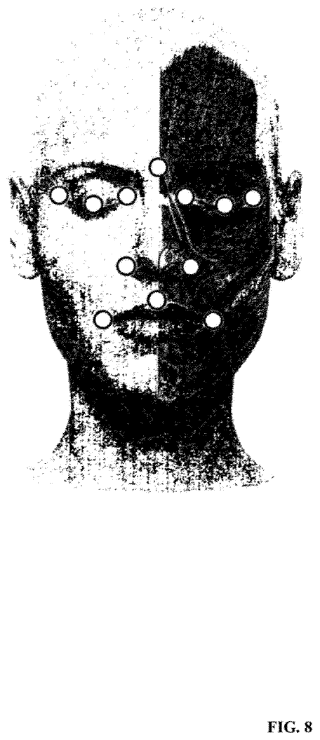

The algorithm of the processor 208 provides a coordinate capture of not less than 12 control points from the projection of the main sensory and motor cranial nerves (FIG. 8), as well as their vector variation. During the consultations, the algorithm allows you to detect emotionally conscious and unconscious micromimic facial expressions of a person, which can be used in the detection system to assess the reliability of perceived and transmitted information, as well as when using test tasks.

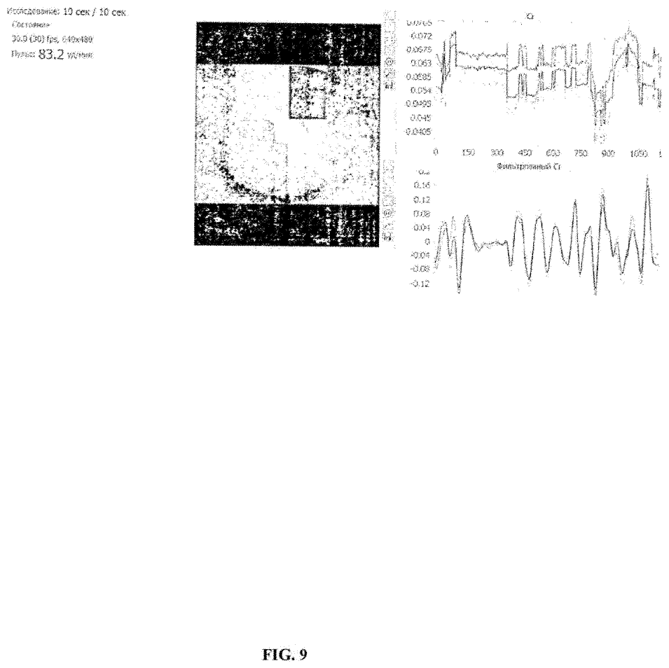

In accordance with the illustration of the diagnostic methods used in the invention, monitoring of the parameters of the amplitude of oscillations of slow physiological waves of a person from any part of the human skin is supported. To this end, the system provides the ability to determine the measurement area ranging from 2 to 400 square centimeters; FIG. 9 shows an example of the registration of parameters of plethysmography from the nail phalanx of a person's finger.

Thus, the claimed invention allows for the early detection of the risks of functional disorders, their correction and monitoring of the effectiveness of preventive measures according to the results of the analysis of the asymmetry of microcirculation of blood through the facial vessels and facial muscle contractions.

1. A telemetry control system for a patient's vital functions containing apparatus for measuring, input and reading of signals, parameters, and graphic data of parameters, including coordinate apparatus based on computer vision technologies that allow monitoring of asymmetric skin color change parameters on areas of the patient's face, a processor connected to the apparatus used for processing the recorded parameters, and an apparatus for displaying the graphic data of the patient's image, the processor operably configured to process the input signals received from at least two projections on a patient's face with a computer program to ascertain amplitude parameters of oscillations of slow physiological waver measuring preset color change coefficients to determine the optical density of tissues, plethysmographic data, and oxygenation parameters.

2. The system, according to claim 1, characterized in that it uses an apparatus for input and reading of signals, parameters, and graphic data of parameters, which is made with the possibility to monitor the skin color gradient with a frequency of at least 30 frames per second.

3. The system, according to claim 1, characterized in that the apparatus for input of graphic data and the processor connected to the apparatus are made with the possibility to use computer vision to ensure the coordinate control of areas of the face and facial contractions.

4. The system, according to claim 1, characterized in that after processing a signal, the processor allows you to determine the main waves to control the patient's vital functions and create a control program based on the principles of biofeedback. The system of the processor provides the ability to output graphic and acoustic signals to influence the patient in order to correct his/her functional state.

5. A method of telemetric monitoring of patient's vital functions comprising:
providing a telemetry control system for a patient's vital functions containing apparatus for measuring, input and reading of signals, parameters, and graphic data of parameters, including coordinate apparatus based on computer vision technologies that allow monitoring of asymmetric skin color change parameters on areas of the patient's face, the telemetry control system having a processor connected to the apparatus used for processing the recorded parameters, and an apparatus for displaying the graphic data of the patient's image, wherein the processor operably configured to process the input signals received from at least two projections on a patient's face with a computer program to ascertain amplitude parameters of oscillations of slow physiological waver measuring;
determining the basic physiological parameters, assessing and analyzing the obtained data, as well as correcting the patient's functional state; these parameters are determined using the graphic information input apparatus and the processor by tracking and recording changes in the color chromaticity gradient and vector changes at the control points of the facial muscles using projections of the patient's face;
at the same time, the patient looks at his/her own image on the display/screen of the computing apparatus with the means for displaying graphical information for a predetermined time, after which the obtained data on the color of the image elements, as well as the color synchronization signal, enter the signal processing system, and the system evaluates the signals taking into account the specified or known coefficients of changes in the chromaticity and coordinates to determine the required parameters.

6. The method, according to claim 5, characterized in that the system records the signals of changes in the color of the image elements and vector changes in the coordinates of the control points in at least 2 areas of the patient's face skin at 12 control points.

7. The method, according to claims 5, characterized in that the patient looks at his/her own image on the display/screen of the computing apparatus designed to display graphic information for at least 300 seconds, which corresponds to the minimum time interval required for assessing human heart rate variability.

8. The method, according to claims 6, characterized in that the data received from the input apparatus and processed by the processor's program allow to determine the amplitude parameters of the oscillations of slow physiological waves of humans with high accuracy: pulse waves, respiratory waves, as well as waves that are caused by parasympathetic or sympathetic cholinergic influences, the myogenic activity of microvascular myocytes, the influence of sensory peptidergic nerve fibers on neuropeptide myocytes, the low-frequency rhythm of impulses of sympathetic adrenergic vasomotor fibers, and the effects of endothelial nitric oxide.

9. The method, according to claim 6, characterized in that the recording of signals related to changes in the color of the elements of the image of the patient's face skin and vector changes in the coordinates at control points from the projection of the patient's face makes it possible to determine blood perfusion in areas of no more than 2 square centimeters.

10. The method, according to claim 6, characterized in that the recording of parameters related to changes in the color of image elements and vector changes in the coordinates at control points from the projection of the patient's face allows to identify the asymmetry of parameters: amplitudes of oscillations of slow physiological waves and manifestations of emotionally conscious and unconscious micromimic facial expressions that may be used by the system of telemetric detection to assess the reliability of transmitted information.
